(19) United States
(12) Patent Application Publication
(10) Pub. No.: US 2014/0010903 A1
(43) Pub. Date: Jan. 9, 2014

(54) Curcuminoid composition with enhanced bioavailability and a process for its preparation

(71) Applicant: Akay Flavours & Aromatics Pvt Ltd., Cochin (IN)

(72) Inventors: Krishnakumar Illathu Madhavamenon, Cochin (IN); Maliakel Balu Paulose, Cochin (IN)

(21) Appl. No.: 13/933,167

(22) Filed: Jul. 2, 2013

(30) Foreign Application Priority Data
Jul. 4, 2012 (IN) 2686/CHE/2012

(51) Int. Cl. A61K 31/12; A61K 47/46; A61K 36/9066

(52) U.S. Cl. CPC A61K 31/12; A61K 36/9066; A61K 47/46
USPC 424/756

(57) ABSTRACT

The invention relates to a curcuminoid composition with enhanced bioavailability derived from fresh turmeric rhizomes and a process for its preparation.

### FIELD OF INVENTION

The invention relates to a curcuminoid composition, derived from fresh turmeric rhizomes, with enhanced bioavailability and a process for its preparation.

### BACKGROUND OF INVENTION

Curcumin, a hydrophobic polyphenol derived from the rhizome of the herb Curcuma longa has a wide spectrum of biological and pharmacological activities. Chemically, curcumin is a bis-R-unsaturated diketone, commonly referred to as diferuloylmethane. Curcumin is generally isolated from dried turmeric rhizomes by solvent extraction with 90 to 95% purity, as a mixture of curcuminoids namely, curcumin, demethoxy curcumin and bisdemethoxy curcumin. Modem scientific research on curcumin with 90 to 95% purity has shown to possesses diverse pharmacologic effects including anti-inflammatory, antioxidant, antiproliferative and antiangiogenic activities. Curcumin is safe even at high doses (12 g/day) in humans but exhibits poor bioavailability. Major reasons contributing to the low plasma and tissue levels of curcumin appear to be due to poor absorption, rapid metabolism, and rapid systemic elimination. Most curcumin is never absorbed and simply passes through the GI tract and is excreted (Anand et al, Molecular pharmaceutics, 2007, 4(6), 807). When taken orally, only traces of curcumin appear in the blood, whereas most of the dose is excreted through the faeces. Sharma et.al. (Clin. Cancer Res 2004, 10(20), 6847) states that most curcumin activities require 0.1-2 micromolar levels in vitro, but current supplements result only in negligible, low nanomolar blood levels.

Curcumin 95% is the most common form of curcuminoid extract composition available in the market. This is a concentrate of 95% curcuminoids, but the bioavailability of the composition is very poor. The compositions derived from dry turmeric with varying percentage of curcuminoid content, and without any excipients or formulation are poorly absorbed in general.

To improve the bioavailability of curcumin, numerous approaches have been undertaken. These approaches involve use of adjuvant like piperine that interferes with glucuronidation; use of liposomal curcumin; use of curcumin nanoparticles; use of curcumin phospholipid complex; and use of structural analogues of curcumin, Despite the lower bioavailability, preclinical studies have established the therapeutic efficacy of curcumin against various human diseases, including cancer, cardiovascular diseases, diabetes, arthritis, neurological diseases and Crohn's disease. Enhanced bioavailability of curcumin is likely to bring this promising natural product to the forefront of therapeutic agents for prevention and/or treatment of human disease.

US19985744121 had observed that administering piperine along with curcumin enhances the bioavailability of curcumin. However the level of enhancement was only modest and no curcumin could be detected after 3 hours even when supplemented with piperine. Moreover, piperine was shown to be toxic in animal studies.

US20070148263 relates to a formulation of curcuminoid with the essential oil of turmeric to enhance the bioavailability of curcumin and to augment the biological activity of curcumin. The disadvantage of this invention is that this formulation is also highly hydrophobic, insoluble, has strong turmeric oil aroma and acts by inhibiting glucuronidation, an important in vivo detoxifying mechanism.

WO2007103435 discloses a formulation comprising curcumin, an antioxidant, a water solubilizing carrier, and optionally a glucoronidation inhibitor forming a curcuminoid-lipid micelle and finally provides as an emulsion or solid-lipid nanoparticles. The formula claims to have 5-times bioavailability in animals upon 100 mg/kg level supplementation, as compared to standard curcumin. This formula containing only 2 to 3% of curcumin claims to have more than 5 times bioavailability upon 100 mg/kg level supplementation, Moreover, the clinical safety of nanoparticles for consumption has not been finalized so far.

WO2007101551 discloses phospholipid complexes of curcumin for improved bioavailability, and WO2010013224 A2 describes the formation of chitosan bound curcumin nanoparticles with an enhancement of 10-fold bioavailability upon oral administration to rats. However, the process and recommended dilution during its preparation, limits its commercial scale preparation.

Sasaki H et al, (Biol. Pharm. Bull., 2011, 34(5), 660-665) discloses colloidal nano particle suspension of curcumin in water comprising gum ghatti and glycerine as additives, prepared using a high pressure homogenization technique. Oral administration of the suspension was reported to enhance the bioavailability by 27 times. However, special equipments are required for its formulation along with chemical additives and non GRAS hydrocolloids. In addition, the reason for enhanced bioavailability has been attributed to the nano form which is difficult to achieve always in commercial scale. The toxicity related to nanoparticles also have not been finalized yet.

All of the above prior art references make use of dry turmeric rhizomes as the starting materials for their preparations. The prior art references teaching the use of fresh turmeric juice is very much limited. US 2010/0015260 A1 provides a method for drying fresh turmeric at 40 to 45° C., followed by powdering and ethanol/water extraction to obtain an extract for the treatment of hyperuricema. US Patent No. 2003/6521271 B1 describes an extraction of dried turmeric rhizomes and its formulation with hydroxyl acids for skin health. The patent also mentioned that the extract powder of fresh rhizomes can also be used for this purpose. However, no information about juicing, its characterization, bioavailability, and benefits were discussed in these documents.

All prior art methodologies involve drying of the fresh turmeric rhizomes followed by the solvent extraction of curcuminoids to highest purity level and further its formulation into bioavailable forms using excipients, adjuvants, and nanoparticle formulation etc. These processes employ organic solvents like hexane, acetone, ethylacetate, ethylene dichloride, methanol etc and final removal of these solvents to zero ppm (parts per million) level is often difficult to achieve.

The present invention was aimed to derive full benefits from the administration of curcumin in human subjects by enhancing its solubility, absorption into plasma and tissues, and by improving its antioxidant and anti-inflammatory potential in vivo upon oral administration at convenient dosages as low as 100 mg/dose.

The present invention relates to cost effective, easy and efficient preparation of curcuminoid composition employing “fresh turmeric” rhizomes which has enhanced bioavailability, is nontoxic and is safe to use. The curcuminoid compositions of the present invention retain the original characteristics of curcumin derived from the natural extracts such as aqueous solubility, stability, hydrophobicity, crystallinity etc.

The present invention has the advantage of providing hydrophobic curcuminoids entrapped in the natural matrix of polysaccharides and proteins of turmeric rhizomes to form a completely water soluble and stable form of curcuminoids without using none of the excipients or additives. The curcuminoid composition thus derived will have food ingredient, dietary supplement, nutraceutical, cosmeceutical and pharmaceutical applications.

### SUMMARY OF INVENTION

In one aspect, the present invention provides a highly bioavailable composition of curcuminoids obtained from fresh turmeric rhizomes comprising:

a) 2 to 10% curcuminoids,

b) 5 to 20% proteins,

c) 50 to 75% carbohydrates

d) 10 to 20% dietary fiber and

e) 1 to 5% of natural oils (hexane extractable).

Wherein each of the components is naturally derived from the fresh turmeric rhizome.

In another aspect, the present invention relates to a process for the preparation of the curcumoid composition having high bioavailability comprising the steps of:

a) Providing fresh rhizomes of turmeric,

b) optionally treating the rhizomes with an enzyme,

c) extracting the fresh juice from the rhizomes,

d) drying the extracted juice to yield a concentrate.

Further scope and applicability of the present invention will become apparent from the detailed description given hereinafter. However, it should be understood that the detailed description and specific examples, while indicating embodiments of the invention, are given by way of illustration only, because various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

### BRIEF DESCRIPTION OF DRAWINGS

The following detailed description of the invention will be better understood when read in conjunction with the appended drawings. For the purpose of assisting in the explanation of the invention, they are shown in the drawings embodiments which are presently preferred and considered illustrative. It should be understood, however, that the invention is not limited to the precise arrangements and instrumentalities shown therein.

FIG. 1 HPLC trace of curcuminoids found in (A) standard regular curcumin with 95% purity isolated from dried turmeric rhizomes (B) fresh turmeric juice powder

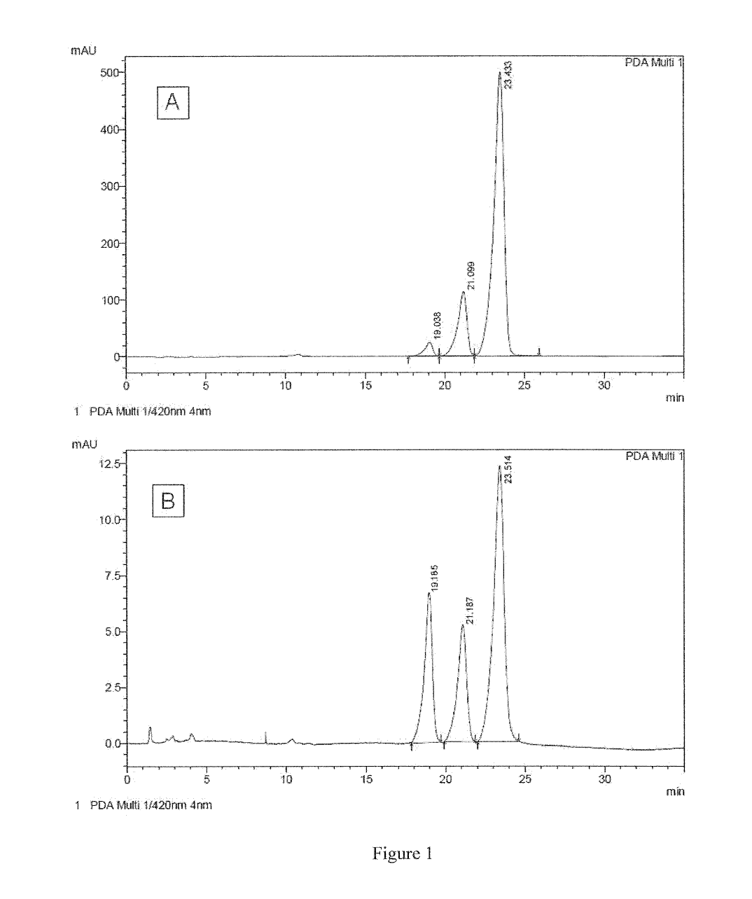

FIG. 2 Average concentration of curcumin observed in human plasma after oral administration of fresh turmeric rhizome juice powder containing 5.2% curcuminoids at doses of 100, 250 and 1000 mg. Regular curcumin with 95% purity was administered at 1000 mg dose. The data is expressed as the mean±SD (n=15); *p<0.05 and **p<0.01, (100 mg, 250 mg and 1000.0 mg fresh rhizome juice powder vs 1 g normal curcumin). Concentration was determined by HPLC/PDA detection.

FIG. 3 Differential Scanning Calorimetry (DSC) of fresh turmeric rhizome juice powder

FIG. 4 Powder X-ray diffraction (PXRD) spectra of fresh turmeric rhizome juice powder

FIG. 5 Scanning Electron Micrograph (SEM) of fresh turmeric rhizome juice powder

### DETAILED DESCRIPTION

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification are to be understood as being modified in all instances by the term “about”. It is noted that, unless otherwise stated, all percentages given in this specification and appended claims refer to percentages by weight of the total composition.

Thus, before describing the present invention in detail, it is to be understood that this invention is not limited to particularly exemplified systems or process parameters that may of course, vary. It is also to be understood that the terminology used herein is for the purpose of describing particular embodiments of the invention only, and is not intended to limit the scope of the invention in any manner.

The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

It must be noted that, as used in this specification and the appended claims, the singular forms “a,” “an” and “the” include plural referents unless the content clearly dictates otherwise. Thus, for example, reference to a “polymer” may include two or more such polymers.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the terms “comprising” “including,” “having,” “containing,” “involving,” and the like are to be understood to be open-ended, i.e., to mean including but not limited to.

As used herein, the term “curcuminoid” intends to mean natural polyphenols that are responsible for the yellow color of turmeric. Curcumin can exist in tautomeric forms, including a 1,3-diketo form and two equivalent enol forms. Curcumin is the principal curcuminoid of turmeric rhizome. Turmeric's other two curcuminoids are demethoxycurcumin and bis-demethoxycurcumin.

As used herein the term “fresh rhizome” intends to mean the green turmeric rhizomes, either harvested from the plants or stored under suitable conditions without losing the moisture content or preventing microbial growth, which are not subjected to any drying such as sun drying, mechanical drying, etc and contains not less than 70% w/w of moisture content, preferably greater than 80% w/w moisture, most preferably greater than 90% w/w of moisture.

The present invention provides a unique composition of curcuminoids obtained from fresh turmeric rhizomes comprising:

a) 2 to 10% curcuminoids,

b) 5 to 20% proteins,

c) 50 to 75% carbohydrates

d) 10 to 20% dietary fiber

e) 1 to 5% of natural oils (hexane extractables).

Wherein each of the components are naturally derived from the fresh turmeric rhizome.

The curcuminoid component of the composition consists of curcumin, demethoxy curcumin and bisdemethoxy curcumin. In an embodiment, the ratio of curcumin ranges between 50 to 78% , the ratio of demethoxy curcumin ranges between 10 to 22% and the ratio of bisdemethoxy curcumin ranges between 5 to 24% when the fresh turmeric rhizome used is curcuma longa. These ratios may differ depending on the species and variety of turmeric rhizome used. But, the ratio of curcuminoids remains as curcumin 72 to 78% , demethoxy curcumin ranges between 10 to 18% and bisdemethoxy curcumin ranges between 3 to 8%, when the normal curcumin derived from dried curcuma longa rhizomes was considered.

The proteins present in the composition are natural proteins and peptides present in turmeric rhizomes. These include all types of proteins present in the turmeric rhizomes, including turmerin, a water soluble peptide, turmeric antioxidant proteins, antifungal proteins, haemaglutinating proteins (lectins) and other unidentified proteins. Turmerin and antioxidant proteins were shown to possess powerful antioxidant properties, which could inhibit lipid peroxidation and scavenge free radicals. The percentage of total protein content in the final composition may range from about 5 to about 20% to the total weight of the composition, depending on the species, variety, geographical location, juicing or extraction method etc.

The carbohydrates present can be natural sugars present in the turmeric rhizomes which are polysaccharides, or sugars. The percentage of carbohydrates present in the final composition may range from about 50 to about 80% to the total weight of the composition.

The natural oils present include the health benefiting essential oils and fixed oils containing components such as termerone, curlone, curumene, cineole, and p-cymene etc. The percentage of the natural oils present in the final composition may range from about 1 to about 5% to the total weight of the composition.

The turmeric composition may also additionally contain anti-oxidants, dietary fiber, many essential vitamins such as vitamin C (ascorbic acid), pyridoxine (vitamin B6), choline, niacin, and riboflavin, etc, and very good amounts of minerals like calcium, iron, potassium, manganese, copper, zinc, and magnesium.

The compositions of the present invention are water soluble, and are impregnated with natural turmeric rhizome derived protein and carbohydrate matrix which help in better absorption. They have improved enhanced efficacy with respect to its in vivo antioxidant, anti-inflammatory, anti-arthritic, anti-amyloid, and anti-ischemic, antiallergic, immunomodulatory etc properties and ability to deposit in the organ tissues such as intestine, liver, spleen, and brain.

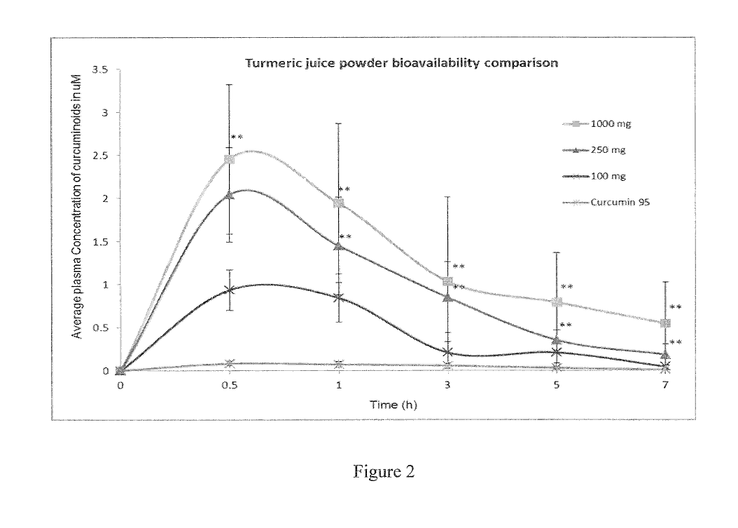

Curcumin from the composition of the present invention is absorbed 30 to 40 times greater than the absorption from standard curcuminoid compositions. Maximum absorption of curcuminoids from the present composition in the plasma was observed around 60 minutes as compared to standard curcumin which was within 30 minutes and enhances the concentration of curcumin in the blood for longer duration upon oral administration (FIG. 2). Moreover, the absorbed curcuminoids stays in the blood stream for longer duration, as evident from the T_{1/2} values of 3.1 hour and C₂₄max of 0.62 μM, as compared to regular curcumin with 95% purity which degraded within 45 min and cannot be seen after 24 hour in the blood plasma (Table 6).

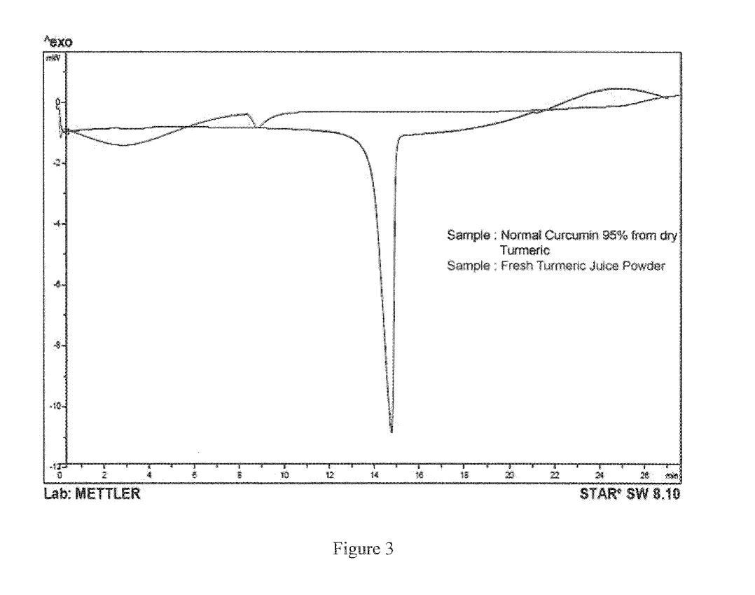

The composition of the present invention can be in the form of a liquid concentrate or a solid powder. When the composition of the present invention is in a dry powder form, the molecular binding, nature of entrapment, surface topography, and stability of curcuminoids in the curcuminoid composition of the present invention can be characterized by DSC, PXRD and SEM analysis. (FIG. 3, 4, 5).

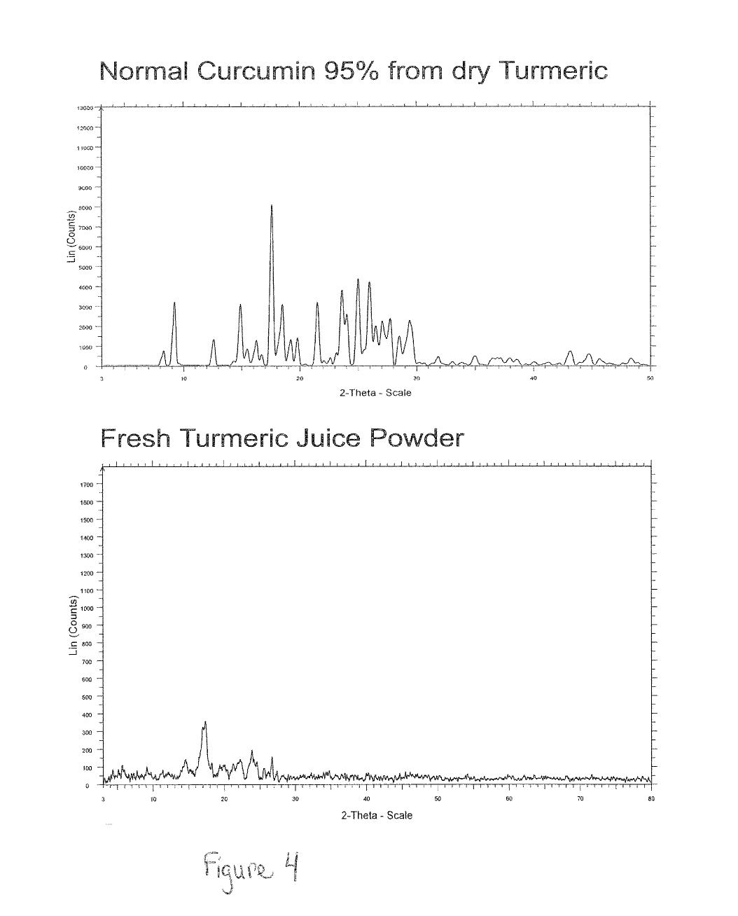

DSC studies (FIG. 3) of the juice powder showed no endothermic peak, indicating the amorphous nature. PXRD studies (FIG. 4) also showed no sharp intense peaks between 7 and 27 degree 2θ indicating typical of amorphous character, with just a broad less intense peak at 16.5° 2θ scattered angle. Thus it is clear that a considerable reduction in crystallinity of curcumin was occurred in juice matrix confirming a kind of the encapsulation effect of the curcuminoids in the complex juice matrix comprising proteins, carbohydrates, dietary fiber etc.

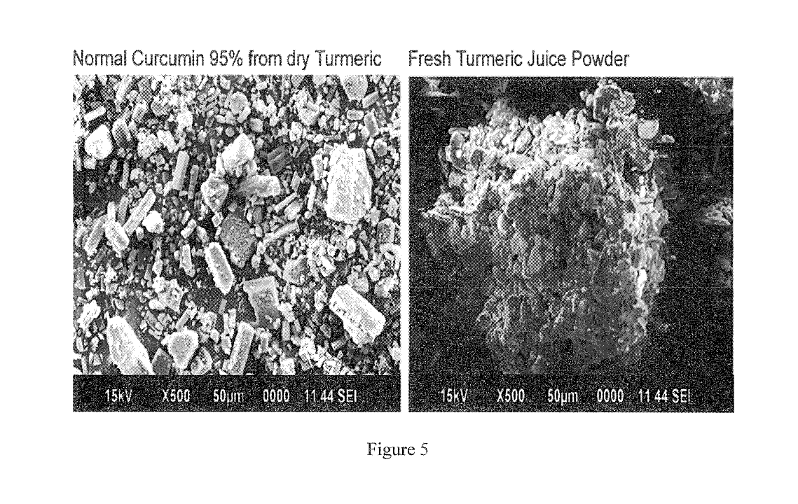

This was further evident from SEM photographs (FIG. 5), the juice powder formed smooth, continues, highly porous microspheres in which crystalline curcumin was uniformly encapsulated. Regular Curcumin 95% on the other hand is a 98% crystalline powder, as evident from above figures.

The curcuminoid composition of the present invention is nontoxic, stable, water soluble and has enhanced bioavailability. The composition does not have any additional excipients or additives, hence making it of organic quality for use in food ingredients, as dietary supplement, or as a nutraceutical, cosmeceutical and pharmaceutical product.

Another object of the present invention is to provide a process for making the curcuminoid compositions with enhanced bioavailability using the juice of fresh turmeric rhizomes without using any excipients or additives and converting to a value-added product for maintaining the health of human beings upon oral consumption.

The process comprises the steps of:

a) Providing fresh rhizomes of turmeric,

b) optionally treating the rhizomes with an enzyme,

c) extracting the fresh juice from the rhizomes,

d) drying the extracted juice to yield a concentrate;

Suitably, fresh, green undried turmeric rhizomes either immediately after harvesting from the plants or stored under suitable conditions without losing the moisture content or preventing microbial growth, which are not subjected to any drying such as sun drying, mechanical drying, etc are used for the purposes of the present invention. Partial drying may also produce enhanced bioavailable powder than the regular curcumin 95%. However, drying can decrease the bioavailability, and yield of the product.

Suitably, various genotypes of curcuma longa rhizomes containing various percentage of curcuminoids can be used to provide the extracted juice containing different levels of curcuminoids ranging between about 2% to about 10% as juice powder, or from about 2% to about 20% as various extracts such as aqueous extract, hydro-alcoholic extract or hydro-acetone extract. Preferably, the rhizomes containing 3% to 12% of curcuminoid content are used for preparing the compositions of the present invention.

Curcuma rhizomes can be selected from the species of Curcuma such as C. longa, C. aeruginosa, C. amada, C. aromatica, C. brog, C. caesia, C. malabarica, C. rakthakanta C. sylvatica, and C. zedoaria. Curcuma longa being the most commonly utilized species, as well as C. zedoaria, and C. xanthorrhiza possesses a wide range of medicinal properties. The relative percentage of total curcuminoids and their percentage in the composition with respect to curcumin, demethoxycurcumin and bisdemthoxy curcumin depends on their presence in the fresh raw material.

Fresh turmeric rhizomes are suitably washed with water to remove mud and sand, stones etc and then sliced with a cutter prior to passing through the extraction procedure.

The extraction of juice is carried out using methods such as mechanical pressing like hydraulic press, screw press, ultrasonication, microwave irradiation or enzymatic treatment or combinations thereof to ground fresh tuimeric rhizomes under specified conditions techniques which aid in obtaining maximum curcuminoids from the rhizome. The extraction process does not involve the use of any organic solvents or other chemicals, and hence, the extract obtained is of organic quality without using any additive/excipients. The extraction is carried out without affecting the natural binding of curcuminoids with the turmeric proteins, dietary fiber and polysaccharides in the natural matrix.

Suitably, the methods of juicing include but not limited to techniques such as mechanical juicing using hydraulic press, screw press etc, grinding the rhizomes followed by ultrasound treatment and mechanical pressing, grinding the turmeric rhizomes and further treatment with enzymes followed by juicing and application of microwave to the grinded mass followed by juicing etc. These methods result in extraction of maximum juice from the fresh rhizomes containing the desired phytochemicals.

In an embodiment, the slices of fresh turmeric rhizomes can be grinded in a wet grinder and transferred to a vessel fitted with an agitator, chilled water jacket, and sonotrode immersed into the grinded paste to provide ultrasonication. Ultrasound can be provided with the help of an ultrasound generator at pulses of duration 1 to 5 min, keeping the temperature less than 50° C. for 20 to 60 min. The paste becomes free flowing and pressed into the juicer to get the juice containing the desired curcuminoids. Ultrasonication enhances the percentage of extraction of the active principle, curcuminoids from the rhizomes.

In another embodiment, the slices of fresh turmeric rhizomes can be grinded in a wet grinder and transferred to a vessel fitted with an agitator and steam jacket and desired levels of enzymes such as pectinase, amylase and protease are added at 0.1 to 0.5%. (w/w) of the grinded mass. The grinded paste can then be stored for appropriate time at appropriate pH and temperature conditions for each enzymes to activate. Finally the paste can be pressed to juice and converted to powder as before. Enzyme treatment is found to enhance the juice yield, clarity of juice and curcuminoid concentration in the juice. Further ultrasonication after enzyme treatment is also found to be effective for enhancing the curcuminoid concentration in the resulting juice

In yet another embodiment, fresh turmeric rhizomes can be ground and the pasty mass can be extracted with ethanol/water mixtures of varying composition, say 10 to 100% v/v and also using 100% water. The liquid extract can then be evaporated to dryness under vacuum and reconstituted with water and spray dried to free flowing powder.

The obtained juice extract can be dried to a convenient water soluble, stable powder/liquid form suitable for oral administration with and/or without using any excipients/additives. The conversion of juice to powder form can be achieved by methods that do not damage the phyto-constituents of the juice matrix including, but not limited to freeze drying, spray drying, vacuum drying, radiant zone drying, and various thin film evaporation techniques and by employing radiations such as infrared.

In an embodiment, the juice is concentrated and spray dried or freeze dried to free flowing powder. The powder contains proteins, peptides, carbohydrates, dietary fiber and curcuminoids along with vitamin C, vitamin B6, Calcium and Iron.

The powder thus obtained can be directly used for its antioxidant, anti-inflammatory activities, and can also be used for bioavailability studies in animals and humans without any further processing.

Yet another object of the present invention is to provide a commercially viable process for the isolation of curcuminoids with more than 95% purity and better yield, from the residue of fresh turmeric rhizomes left after juice extraction for maximum utilization of turmeric rhizomes. The process of extraction of juice from the residue comprises drying the residue obtained after extraction of the juice and subsequent extraction of residue to isolate curcuminoids with more than 90% purity level. The extraction of juice from the residue can be carried out using techniques known in the art such as solvent extraction procedures and the like.

About 85% w/w of the plant material is left as residue of the juice extraction process which may contain more than 85% of the active principle which has not been extracted in juice. The residue thus left after juice extraction can be suitably processed as per the existing methods for extracting the desired phytochemicals as per the methods known in the prior arts. The residue left after juice extraction can be dried over sun and extracted with a mixture of hexane and acetone and solvent was evaporated to get a pasty mass called turmeric oleoresin. Other solvents like ethyl alcohol, ethyl acetate, ethylene chloride or their mixtures thereof can also be used for the extraction. The oleoresin can be dissolved in varying compositions of ethanol, isopropanol, ethylacetate or mixtures thereof to crystallize curcuminoids with more than 90% purity, and was filtered and dried under vacuum at 70° C. The extract can be pulverized to get curcumin powder of 90-95% purity with an average particle size of 150-250 microns.

Purity of the curcuminoid powder composition thus obtained can be determined spectrophotometrically using the method of ASTA 2005, by measuring the absorbance at 420 nm in acetone. (Ref: Official analytical methods of the American Spice Trade association, 4^{th} edition, 1997, New Jersey, pp 81-83). Individual curcuminoids are detected and estimated with HPLC/PDA detection method, employing earlier reported procedure (Jadhav B K, et al. Chromatographia 06-00303; Manuscript number 0164).

The enhanced bioavailability or efficacy depend linearly on the curcumin content in the juice and may vary according to the nature of cultivar, species, climatic and soil conditions where it is grown, juicing methods etc.

The compositions of the present invention are physiologically stable and directly compressible to a non-hygroscopic powder form for easy handling and formulation in tablet and capsule forms.

The stability of fresh turmeric juice powder containing curcuminoids was tested by following the FDA guide lines on accelerated stability studies. Stability of the sample was determined by measuring the parameters including color, appearance, odour, curcumin content, moisture, total fat, carbohydrates, dietary fiber and protein content (Table 1) in the stability period. The compositions of the present invention does not show significant loss of nutrients when stored for about 2 years in air tight containers, kept at cool, dark place protected from moisture.

TABLE 1

| | Fresh turmeric juice powder with curcumin content | | | | |
|---|---|---|---|---|---|
| | 5.2% prepared from VAR-1, as example-1 | | | | |
| Parameter | 0 month | 1 month | 2 month | 3 month | 6 month |
| Appearance | Yellowish | Yellowish | Yellowish | Yellowish | Yellowish |
| | brown powder | brown powder | brown powder | brown powder | brown powder |
| Identification | Complies | Complies | Complies | Complies | Complies |
| Odour | Characteristic | Characteristic | Characteristic | Characteristic | Characteristic |
| Curcumin content | 5.2% | 5.2% | 5.1% | 5.1% | 4.9% |
| Moisture | 3.7% | 3.7% | 3.5% | 3.5% | 3.5% |

Nutritional profile

| | | | | | |
|---|---|---|---|---|---|
| Total fat | 3.2 g/100 g | 3.2 g/100 g | 3.2 g/100 g | 3.2 g/100 g | 3.2 g/100 g |
| Total carbohydrate | 59 g/100 g | 59 g/100 g | 59 g/100 g | 59 g/100 g | 59 g/100 g |
| Dietary fibre | 18 g/100 g | 18 g/100 g | 18 g/100 g | 18 g/100 g | 17 g/100 g |
| Protein | 14 g/100 g | 14 g/100 g | 14 g/100 g | 14 g/100 g | 14 g/100 g |

Table-1 Stability data for fresh turmeric juice powder with curcumin content 5.2% under Accelerated testing conditions of temperature 40±2° C. and relative humidity 75%±5%.

Oral administration of newly prepared curcuminoids described in the present invention enhanced the bioavailability at 250 mg/kg dosage in animals (wistar rats) (Table 2 & 3) and human hepatic bioavailability improved at a dose of 1000 mg, 250 mg and 100 mg as compared to standard curcumin dosage of 1000 mg when the area under plasma curcumin concentration-time curve (AUC) was considered (FIG. 2 & Table 6).

The extent of bioavailability may vary from 2 to 40, depending on the juice powder produced. Higher the curcuminoid concentration in the juice, greater the bioavailability and hence the efficacy.

TABLE 2

| | Curcumin | Juice | Curcumin | |
|---|---|---|---|---|
| Name of variety | content in | powder | content in | Bioavail- |
| and geographical | fresh | yield | juice powder | ability |
| location | rhizome | (%)^{a} | (%)^{b} | in animals ^{c} |
| VAR -1, Cambodia | 10.6% | 9.8 | 6.1% | 36.4 |
| VAR -2, India | 6.4% | 7.2 | 3.1% | 24.8 |
| VAR -3, India | 4.7% | 6.3 | 2.3% | 14.9 |
| VAR -4, Burma | 7.2% | 7.5 | 4.2% | 28.9 |
| VAR -5, India | 11.4% | 7.9 | 6.6% | 39.1 |
| VAR -6, India | 10.8% | 8.2 | 5.8% | 34.8 |
| Standard | — | — | 95% | 1 |
| Curcumin 95% | | | curcuminoids | |

^{a}Juice powder yield denotes the weight of juice powder obtained from 100 g of fresh turmeric rhizomes by following a standard procedure of mechanical juicing followed by spray drying without using any additives.
^{b}The curcumin content per 100 g of fresh turmeric juice powder is measured spectrophotometrically by the method of ASTA, by monitoring the absorption maximum at 420 nm
^{c} Bioavailability was measured by quantifying the curcuminoid concentrations (HPLC method) in blood plasma after oral supplementation of turmeric juice powder to adult Wistar rats at 250 mg/kg body weight dosage. The enhancement in bioavailability was then compared with the curcuminoid absorption on standard curcuminoids with 95% purity at same dosage. Area Under Curve (AUC) of plasma curcumin concentration -time plot was calculated as a measure for bioavailability and other pharmacokinetic parameters (Details are given in Example -10).

Table-2 Yield, curcumin content and bioavailability of various varieties of fresh turmeric rhizome juice powder obtained by following the procedure described in Example-1. The juice was expressed by a screw type mechanical press and powdering was carried out by a standardized spray drying technique without using any additives or excipients.

TABLE 3

| | | | | Curcumin | |
|---|---|---|---|---|---|
| Name and | | | Yield of | content of | |
| Loca- | Curcumin | Solvent | extract | extract | Bioavail- |
| tion of | content in | used for | powder | powder | ability |
| variety | rhizomes | extraction | (%) ^{a} | (%) | in animals |
| VAR-1 | 10.6% | Water | 10.2% | 2.9% | 8.6 |
| Cambodia | | Ethanol/water | 9.2% | 6.8% | 14.7 |
| | | (70/30 v/v) | | | |
| | | Ethanol/water | 9.5% | 5.2% | 11.1 |
| | | (50/50 v/v) | | | |
| | | Ethanol/water | 10% | 3.4% | 6.2 |
| | | (30/70 v/v) | | | |
| Curcumin | — | — | — | 95.2% | 1 |
| 95 | | | | | |

^{a} Yield, curcumin content and bioavailability as compared to standard curcumin 95 was calculated using the same protocols described in FIG. 2. Details were given as examples 7 & 8

Table-3 Yield, curcumin content and bioavailability of extracts obtained from fresh rhizomes of turmeric, VAR-1, by various extraction techniques and their observed bioavailability as compared to the regular curcumin 95% isolated from dried turmeric rhizomes by solvent extraction. Briefly, 250 g of ground fresh turmeric rhizomes was extracted with water and ethanol-water mixtures containing various proportions, say 20/80 v/v to 80/20 v/v. The extract obtained was concentrated under vacuum to remove the alcohol and the resulting concentrated water extract was spray dried to powder form.

TABLE 4

| Name, location | | | | |
|---|---|---|---|---|
| and curcumin | | | Curcumin | Bioavail- |
| content of dry | Solvent used | Yield | content in the | ability |
| raw material | for extraction | (%)^{b} | extract (%) | in animals |
| VAR-1, containing | Acetone | 20 | 37.4 | 1.4 |
| 10.6% | Ethanol | 14 | 30.3 | 1.2 |
| curcuminoids | Water | 9.6 | 1.3 | 1.2 |
| Combodia | | | | |
| | Ethanol/water | 15.3 | 2.1 | 1.4 |
| | (70/30 v/v) | | | |
| | Acetone/water | 18.7 | 3.8 | 1.4 |
| | (70/30 v/v) | | | |

^{a} Acetone and ethanol extracts were pasty mass and other extracts were evaporated to remove the solvent and spray dried to powder
^{b}Yield, curcumin content and bioavailability were calculated using the same protocols described in Table-2. Details were given as example 9.

Table-4 Yield, curcumin content and bioavailability of various extract powders of dry turmeric rhizome of variety, VAR-1 (as described in Table-2) prepared using the procedure described in example-3. Briefly, 250 g of ground dry turmeric rhizomes was extracted with water, ethanol, acetone and ethanol-water mixtures 70/30 v/v. The extract obtained was concentrated to remove the solvent under vacuum and the resulting concentrated water extract was spray dried to powder form as before. Acetone and ethanol extract were obtained as pasty mass and used as such.

The present invention can be extended to the extraction of suitable phytochemicals from the various other plants or their parts like for example rhizomes of ginger (Zingiber Officinalis) for gingerols, leaves of Brahmi (Bacopa minniorie) for bacosides, leaves of tea (Centinela sinesis.) for catechins, all areal parts of Gymnema sylverstre for gymnemic acids, fruits of Emblica officianalis for tannins and vitamin C, etc.

Though the relative percentage of extraction of the active principle from the plant part by the application of these techniques may be different for different plants, the methods are generally applicable to natural plant derived raw materials containing greater not less than 75% v/w of moisture, preferably greater than 85%, most preferably greater than 90%. It has been observed that the percentage of active ingredient that can be extracted into the juice is very important for enhanced bioactivity. Higher the percentage, greater the efficacy.

The following examples are provided to better illustrate the claimed invention and are not to be interpreted in any way as limiting the scope of the invention. All specific materials, and methods described below, fall within the scope of the invention. These specific compositions, materials, and methods are not intended to limit the invention, but merely to illustrate specific embodiments falling within the scope of the invention. One skilled in the art may develop equivalent materials, and methods without the exercise of inventive capacity and without departing from the scope of the invention. It is the intention of the inventors that such variations are included within the scope of the invention.

### EXAMPLES

### Example 1

1 Kg of fresh turmeric rhizomes of various turmeric varieties were sliced and pressed through a screw type fresh juicer. The residue left after first juice extraction was again pressed three times. All the juices were combined, centrifuged to remove the debris and finally converted to free flowing powder by spray drying and freeze drying. Bioavailability of each of the juice powder was evaluated in wistar rats. Deatiles of juice yield, curcumin content and bioavailability of different varieties are given in Table 2.

### Example 2

1 Kg of fresh turmeric rhizomes of finger turmeric (coded as, VAR-1) variety was sliced and grinded to a pasty mass and treated with an enzyme pectinase at pH 4.5 to 5.5 45 to 55° C. for 24 hr. Ultrasonication was performed after enzyme treatment as before and juice was expressed. The residue left after first juice extraction was again pressed three times. All the juices were combined, centrifuged to remove the debris and finally converted to free flowing powder by spray drying and freeze drying. Deatiles of juice yield and curcumin content of pectinase treatment is given in Table 5.

### Example 3

1 Kg of fresh turmeric rhizomes of finger turmeric (coded as VAR-1) variety was sliced and grinded to a pasty mass and treated with an enzyme amylase at pH 4 to 5, at 45 to 55° C. for 24 hours. Ultrasonication was performed after enzyme treatment as before and juice was expressed. The residue left after first juice extraction was again pressed three times. All the juices were combined and centrifuged to remove the debris and finally converted to free flowing powder by spray drying and freeze drying. Deatiles of juice yield and curcumin content of amylase treatment is given in Table 5.

### Example 4

1 Kg of fresh turmeric rhizomes of finger turmeric (coded as VAR-1) variety was sliced and grinded to a pasty mass and treated with an enzyme protease at pH 7 to 9, 50 to 60° C. for 24 hours. Ultrasonication was performed after enzyme treatment as before and juice was expressed. The residue left after first juice extraction was again pressed three times. All the juices were combined, centrifuged to remove the debris and finally converted to free flowing powder by spray drying and freeze drying. Deatiles of juice yield and curcumin content of protease treatment is given in Table 5.

### Example 5

1 Kg of fresh turmeric rhizomes of finger turmeric (coded as VAR-1) variety was sliced and grinded to a pasty mass and treated with enzymes in a stepwise manner. First treated with pectinase at 4.5 to 5.5 at 50±2° C., for 8 hours and then with amylase under same conditions for another 8 hours and finally with protease at pH 8.0 at 50±2° C. for another 8 hours. Ultrasonication was then applied as before and juice was expressed. The residue left after first juice extraction was again pressed three times. All the juices were combined, centrifuged to remove the debris and finally converted to free flowing powder by spray drying and freeze drying. Deatiles of juice yield and curcumin content after above mentioned treatment is given in Table 5.

### Example 6

1 Kg of fresh turmeric rhizomes of finger turmeric (coded as VAR-1) variety was sliced and grinded to a pasty mass. It is then subjected to ultrasonication for 30 to 60 min, employing an ultrasound generator of 1000 watts as pulses of duration 1 to 5 min, keeping the temperature below 50° C. The pasty mass after ultrasonication was pressed to juice. The residue left after first juice extraction was again pressed three times. All the juices combined, centrifuged to remove the debris and finally converted to free flowing powder by spray drying and freeze drying. Deatiles of juice yield and curcumin content after ultrasonication are given in Table 5.

TABLE 5

| Name, location and | | | |
|---|---|---|---|
| curcumin content of | | | Curcumin |
| raw material | Technique of extraction | Yield^{a} | content |
| VAR-1, containing | Mechanical press | 9.8% | 6.1% |
| 10.6% curcuminoids | Ultrasonication | 9.3% | 6.8% |
| Combodia | | | |
| | Enzyme treatment using | | |
| | i) Pectinase | 9.3 | 6.1 |
| | ii) Amylase | 9.2 | 6.4 |
| | iii) Amyloglucosidase | 9.4 | 6.2 |
| | iv) Protease | 9.6 | 5.7 |
| | v) Combinations of these | 9.2 | 6.3 |
| | Enzyme treatment followed | | |
| | by sonication | | |
| | i) Pectinase | 9.2 | 6.6 |
| | ii) Amylase | 9.4 | 6.9 |
| | iii) Amyloglucosidase | 9.0 | 6.9 |
| | iv) Protease | 9.2 | 6.3 |
| | v) Combinations of these | 8.9 | 7.2 |

^{a}Yield and curcumin content were calculated using the same protocols described in Table 2.

Table-5 Yield and curcumin content of various extract powders of fresh turmeric rhizome of variety, VAR-1 (as described in Table-2) prepared using various juice expression techniques. Briefly, 250 g of ground fresh turmeric rhizomes was expressed for juice by mechanical means, ultrasonication, enzyme treatment and enzyme treatment followed by ultrasonication. The resulting concentrated juice was spray dried to powder form.

### Example 7

1 Kg of fresh turmeric rhizomes of finger turmeric (coded as VAR-1) variety was sliced and ground with the addition of some water (say, 0.5 to 2 times w/v) and pressed through a screw type fresh juicer. The residue left after first juice extraction was again pressed by adding 1 to 3 times (w/v) of water and repeated three times. All the juices were combined, centrifuged to remove the debris and finally converted to free flowing powder by spray drying and freeze drying. Details of water extract yield and curcumin content are given in Table 3.

### Example 8

1 Kg of fresh turmeric rhizomes of finger turmeric (coded as VAR-1) variety was sliced and ground with the addition of 3 to 5 times w/v of ethanol-water mixture and kept under agitation for 2hr. The solvent was drained to another vessel The extraction was repeated in the same manner for 5 to 7 times and the resulting solvent containing the extractives of fresh turmeric rhizomes were mixed together and evaporated under vacuum, at 50 to 60° C. The pasty mass thus obtained (oleoresin) was uniformly mixed and analyzed for yield and curcumin content. The ratio of ethanol-water was adjusted to 70/30, 50/50, 30/70 to prepare various extracts and the curcumin content of the extracts were also measured. Those having curcumin content greater than 3% was subjected to bioavailability analysis (Table-3).

### Example 9

1 Kg of dry turmeric rhizomes of finger turmeric (CFT) variety was sliced and ground with the addition of 3 to 5 times w/v of ethanol and kept under agitation for 2 hr. The solvent was drained to another vessel The extraction was repeated in the same manner for 5 to 7 times and the resulting solvent containing the extractives of dry turmeric rhizomes were mixed together and evaporated under vacuum, at 50 to 60° C. The pasty mass thus obtained (oleoresin) was uniformly mixed and analyzed for yield and curcumin content. The same extraction was repeated with acetone, water and ethanol/water (70/30 v/v) and acetone/water (70/30 v/v) mixtures and the resulting extracts were analysed (Table 4).

### Example 10

The bioavailability of the various juices, juice powders and extract powders produced in the present invention from both fresh and dried rhizomes of turmeric was tested against the standard curcumin with 95% purity on adult wistar rats of both sexes weighing 180 to 200 g body weight. Rats were fasted overnight and received either normal curcumin or juice powder dissolved or suspended in a suitable aqueous solution containing 0.1% carboxymethylcellulose, at 250 mg/kg body weight by oral gavage. At 30 minute post dose, animals were exsanguinated and blood was collected after each time point, ca. 0, 0.5,1, 3, 5 and 7 hours respectively into heparinized tubes and centrifuged at 6000×g for 15 minutes. Plasma was decanted and stored at −80° C. until analysis. Plasma samples at various time points up to 5 hr post administration were subjected to curcuminoid concentration estimation by HPLC/PDA. Concentration of curcumin in plasma at each time point was calculated as an average of six HPLC measurements using methanol mobile phase which eluted total curcumin (free curcuminoids plus its metabolites) as a single peak at retention time 2.9 min (Ref: US 2007/0148263 and Krishnakumar et al; Journal of Functional Foods, 2012, 4, 348-357). Briefly, 1 mL of plasma was extracted with 3×10 mL of 1:1 v/v of ethylacetate and was dried over anhydrous sodium sulphate and evaporated to dryness. The residue was then made up to 10 mL with methanol and 20 μL was injected to Shimadzu model SPD-20 A HPLC fitted with a PDA detector model M20A using a reverse phase C10 column (Phenomenex 250×4.6 mm, 5μ and methanol mobile phase monitored at 420 nm.

Measurement of curcumin content in plasma was validated by spiking a standard curcumin (CAS Registry No. 458-37-7) in animal blood and plasma at 1.0 μg/mL. Curcuminoid retention time was confirmed by repeated 10 analyses at 50 mg level on same column under identical conditions. Efficiency of extraction from blood and plasma was also confirmed by spiking 1 mg/mL standard curcumin. Range and linearity were determined for curcumin extracted from blood and plasma as mentioned above and the recovery of curcuminoids were calculated to be 89 and 92.1% respectively. Plasma samples at various time points up to 24 hr post administration were subjected to HPLC-PDA analysis, after confirming the detected peak identity using an electrospray ionization mass spectrometer in negative ionization mode, which could detect molecular masses corresponding to protonated curcumin at 368 Analysis was performed by incorporating a Waters Alliance LC fitted with Waters 3100 ESI mass detector. The relative enhancement in bioavailability of various extracts and juice powders produced in the present invention. as seen from the area under curve (AUC_{0-7 h}) calculations of the plasma curcumin concentration−time plot was shown in Tables 1,3 and 4. The pharmacokinetic parameters like the maximum concentration of curcuminoids found in the plasma (Cmax), the time at which maximum plasma concentration of curcuminoids was found in the blood stream (Tmax), the time taken for 50% of absorbed curcumin to degrade half of its maximum concentration (T_{1/2} max) were also estimated. It was found that the bioavailability of the fresh rhizome juice powders are increased to 6 to 38 times depending on the curcumin concentration obtained in the juice powder, irrespective of the method of juice expression. Higher the curcumin content, higher the bioavailability (Table 1, 3 and 4). Ethanol, water and ethanol/water mixture extracts of dry turmeric rhizomes on the other hand produced only marginal enhancement in bioavailability, say less than 2% as compared to standard curcumin 95% (Table 4).

### Example 11

The enhancement of bioavailability was also tested on human volunteers (both sex) of age group between 25 to 50, who were healthy and not involved in any medication or health supplementation. The volunteers were not allowed to take turmeric-containing food for two days prior to the test. The juice powders which showed maximum bioavailability enhancement upon animal studies are selected for human studies. 15 volunteers were given 100 mg (100 mg×1), 250 mg (250 mg×1) and 1000 mg (500 mg×2) of fresh turmeric rhizome juice powder containing curcuminoids. 5 mL blood of each volunteer was withdrawn at 0, 0.5, 1, 3, 5, 7 and 24 hours respectively and plasma samples were deep-frozen at −80° C. till analysis. 1 mL of the plasma was then analysed for curcumin content by HPLC as was done for animal studies. After one week of juice powder feeding, the volunteers were also given 1000 mg (500 mg×2) of normal standard curcumin 95% and protocol was repeated exactly the same for collection and analysis of blood samples at various time intervals. The relative enhancement in bioavailability of various juice powders produced in the present invention is depicted in FIG.-2 and the area under curve (AUC_{0-7 h}) calculations of the plasma curcumin concentration−time plot was considered as the measure of bioavailability. The pharmacokinetic parameters like the maximum concentration of curcuminoids found in the plasma (Cmax), the time at which maximum plasma concentration of curcuminoids was found in the blood stream (Tmax), concentration of curcuminoids found in the plasma after 24 hours (C₂₄max) and the time taken for 50% of absorbed curcumin to degrade half of its maximum concentration (T_{1/2}max) were also estimated (Table-6). It was found that the bioavailability of the fresh rhizome juice powders are increased to 6 to 41 times depending on the curcumin concentration obtained in the juice powder, irrespective of the method of juice expression. Higher the curcumin content, higher the bioavailability (FIG. 2 and Table 2).

TABLE 6

| | | | | C24 | | T½ | Bioavailability |
|---|---|---|---|---|---|---|---|
| Sample | Subjects | Dose | Cmax | max | Tmax | max | in number |
| Administered | (n) | (g) | (uM) ^{a} | (uM) | (h)^{b} | (h) ^{c} | of times ^{e} |
| VAR-1 Turmeric | 15 | 100 mg/kg | 1.1 | 0.31 | 0.65 | 2.8 | 20.4 |
| juice powder | | | | | | | |
| VAR-1 Turmeric | 15 | 250 mg/kg | 2.4 | 0.47 | 0.65 | 2.8 | 28.9 |
| juice powder | | | | | | | |
| VAR-1 Turmeric | 15 | 1000 mg/kg | 3.3 | 0.62 | 0.65 | 3.1 | 41.2 |
| juice powder | | | | | | | |
| Normal | 15 | 1000 mg/kg | 0.08 | — | 0.5 | — | 1 |
| curcumin 95 | | | | | | | |

^{a} Cmax, is the maximum concentration of curcuminoids observed in the blood plasma of adult wistar rats after oral supplementation of fresh turmeric juice powder and normal standard curcumin and C₂₄ max is the curcuminoid concentration found after 24 h post-administration. Similarly, Tmax is the time point at which maximum curcumin concentration has been found in the blood plasma, T_{1/2} max is the time taken to degrade the observed maximum concentration of curcuminnoids to half its value, Bioavailability was estimated from Area Under Curve (AUC) calculations as compared to standard curcumin. C₂₄max and T_{1/2} max for Normal curcumin 95 could not calculate as it is not detected.

Table-6 Pharmacokinetic parameters calculated from plasma curcumin concentration−time plot for human volunteers, after administration of turmeric juice powder obtained from VAR-1, explained in example 10 & 11

### Example 12

Standard curcumin (CAS Registry No. 458-37-7) was purchased from Sigma-Aldrich, Bangalore, India. All solvents and reagents for analysis were of HPLC grade and that for extraction were of analytical grade from Merck, Mumbai, India. MilliQ Plus (Millipore) purified water was employed for all experiments. Sonication was performed using 1000 W ultrasound generator fitted with a sonotrode (Hieilscher, Germany). UV/VIS analysis was performed on Varian-Cary 5000 UV-VIS-NIR spectrophotometer (Varian Inc, Chennai, India). Thermogram was recorded using Q10 DSC, differential scanning calorimeter (DSC Metier Toledo 822e), Mettler-Toledo India Pvt Ltd, Mumbai, India. Samples (3-5 mg) were sealed in the aluminum crimp pan, and heated at a rate of 10° C./min from 30 to 300° C. under a nitrogen atmosphere. Powder X-ray diffraction studies (PXRD) were performed on a Bruker D8 Advance instrument: target Cu, λ-1.54 A°, filter-Ni, voltage 40 kV, time constant 5 min/s; scanning rate 1°/min (Bruker AXS GmbH, Karlsruhe, Germany). Scanning electron microscopic analysis was done on SEM Jeol 6390 LA equipment (JEOL Ltd, Tokyo, Japan). HPLC analyses were carried out on a Shimadzu LC 20 AT system, with M20A Photo diode array (PDA) detector ((Shimadzu Analytical Pvt ltd, Mumbai, India), fitted with a reverse phase C18 Phenomenex column (250×4.6 mm, 3μ) using methanol as the mobile phase. LC/MS was performed on Waters HPLC (Alliance system, with MassLynx software) fitted with 3100 ESI mass detector (Waters Ltd, Bangalore, India).

Stability and crystallinity were assessed by differential scanning calorimeter (DSC), and Powder X-ray diffraction (PXRD), investigations of standard curcumin 95 and juice powders exhibiting enhanced bioavailability. DSC studies (FIG. 3) showed a sharp endotherm at 186° C. for pure curcumin due to its melting, whereas the juice powder showed no endothermic peak, indicating the amorphous nature. PXRD studies (FIG. 4) of curcumin showed sharp and intense peaks between 7 and 27° 2θ, whereas the juice powder gave a pattern typical of amorphous character, with just a broad less intense peak at 16.5° 2θ scattered angle. Thus it is clear that a considerable reduction in crystallinity of curcumin was occurred in juice matrix confirming a kind of the encapsulation effect of the curcuminoids in the complex juice matrix comprising proteins, carbohydrates, dietary fiber etc. This was further evident from SEM photographs (FIG. 5). Curcumin showed various shapes with well-defined edges whereas the juice powder formed smooth, continues, highly porous microspheres in which crystalline curcumin was uniformly encapsulated.

### Example 14

1 Kg each of fresh raw materials of ginger rhizomes, total ariel parts of bacopa miniorie, gymnema sylvestre leaves, and emblica officianlis fruits were juiced using an IKA make kitchen juicer and the active ingredients levels in the resulting powder was estimated Just like in the case of fresh rhizomes of turmeric, the juice yield, percentage of active principles in the juice etc can be improved by selecting the various varieties of raw materials from different geographical locations and expressing the juice with the help of techniques such as enzymes or ultrasonication. Since a detailed informations of all these informations on each of the plant type is beyond the scope of this invention, the present invention illustrated such effects in detail for turmeric rhizomes and curcuminoids. The residue left after first juice extraction can be further dried using the methods known in the prior arts for bulk solvent extraction and to isolate the phytochemical of interest. Thus the present invention provides a general procedure for making the juice of paint materials containing sufficient concentration of active ingredient for better bioavailability and efficacy. In the usual conventional process of isolation or solvent extraction using dried plant materials, the valuable juice content usually neglected.

We claim:

1. A curcuminoid composition obtained from fresh turmeric rhizomes comprising:
a. 2 to 10% curcuminoids,
b. 5 to 20% proteins,
c. 50 to 75% carbohydrates,
d. 10 to 20% dietary fiber and
e. 1 to 5% of natural oils (hexane extractable),
wherein each of the component is naturally derived from the fresh turmeric rhizome.

2. The curcuminoid composition as claimed in claim 1, wherein the curcuminoid component comprises of curcumin, demethoxycurcumin, and bisdemethoxycurcumin wherein the percentage of curcumin ranges between 50-78%, the ratio of demethoxycurcumin ranges between 10 to 22%, and the ratio of bisdemethoxycurcumin ranges between 5 to 24%.

3. The curcumnoid composition as claimed in claim 1, wherein the composition is obtained from a fresh rhizome of Curcuma longa L.

4. The curcuminoid composition as claimed in claim 1, wherein the percentage of curcuminoids ranges between 3 to 8%, percentage of proteins ranges between 10 to 16%, the percentage of carbohydrates ranges between 60 to 70%, the percentage of dietary fiber ranges between 12 to 18% and the percentage of fixed oils is less than 3%.

5. A process of making a curcuminoid composition as claimed in claim 1 comprising the steps of:
a. providing fresh rhizomes of turmeric,
b. optionally treating the rhizomes with an enzyme,
c. extracting the fresh juice from the rhizomes,
d. drying the extracted juice to yield a concentrate.

6. The process as claimed in claim 5, wherein the rhizomes are selected from the species of Curcuma longa.

7. The process as claimed in claim 5, wherein the curcuminoid content in the fresh rhizome ranges from 3 to 12%.

8. The process as claimed in claim 5, wherein the extraction process in step b) can be aided with the help of ultrasonication or microwave to result maximum extractability for the active principles.

9. The process as claimed in claim 5, wherein the extraction process in step b) can be aided with the help of various enzyme treatments.

10. The process as claimed in claim 9, wherein the enzymes used are amylase, protease or pectinase either alone or in combination.

11. The process as claimed in claims 5 to 10, wherein ultrasonication and microwave irradiation can be performed to enzyme treated rhizomes.

12. The process as claimed in claim 5, wherein the drying of extracted juice in Step d) can be carried out using techniques such as freeze drying, spray drying, vacuum drying, radiant zone drying, and various thin film evaporation techniques or by employing radiations such as infrared or similar technologies known in the prior arts.

13. The process as claimed in claim 5, which comprises isolation of curcuminoids with not less than 95% purity from the residue of fresh turmeric rhizomes left after juice extraction.

14. The process as claimed in claim 13, wherein the isolation of curcuminoids from the residue is carried out using solvent extraction techniques.

15. The curcuminoid composition as claimed in claim 1, wherein the composition is in the form of a liquid concentrate.

16. The curcuminoid composition as claimed in claim 1, wherein the composition is in the form of a dry powder

17. The curcuminoid composition as claimed in claim 1, wherein the composition is amorphous in nature.

18. The curcuminoid composition as claimed in claim 1, wherein the composition provides 6 to 40 times higher bioavailability as compared to the normal curcumin 95% isolated from dried turmeric rhizomes upon oral administration.

19. The curcuminoid composition as claimed in claim 1, wherein a T_{1/2} of the composition is at least 3 hr. Where T_{1/2} is the time taken to degrade to half the concentration of absorbed curcumin in the blood plasma.

20. The curcuminoid composition as claimed in claim 1, wherein the composition provides curcuminoids in blood plasma for longer duration, with a C₂₄max, not less than 0.3 μM.
